(19) United States
(12) Patent Application Publication
(10) Pub. No.: US 2021/0277525 A1
(43) Pub. Date: Sep. 9, 2021

(54) Unlimited Ethanol Based Hand Sanitizer

(71) Applicants: Brandon Iglesias, New Orleans, LA (US); Yang Song, New Orleans, LA (US)

(72) Inventors: Brandon Iglesias, New Orleans, LA (US); Yang Song, New Orleans, LA (US)

(21) Appl. No.: 17/189,901

(22) Filed: Mar. 2, 2021

Related U.S. Application Data
(60) Provisional application No. 62/984,709, filed on Mar. 3, 2020.

(51) Int. Cl. C25B 3/21; C25B 3/07

(52) U.S. Cl. CPC C25B 3/21; C25B 3/07

(57) ABSTRACT

A process provides an unlimited source of ethanol based, liquid disinfectant for application directly to human hands or to hand wipes, by combining a carbon nanospike catalyst with a copper catalyst, carbon dioxide, water and water vapor in an electrochemical process initiated by a power source. Further, an improved process provides for making the carbon nanospike, through injection with photons and electromagnetic waves.

### CROSS REFERENCE TO RELATED APPLICATIONS

Applicants claim priority to Provisional Application No. 62/984,709, which was filed Mar. 3, 2020.

### STATEMENT REGARDING FEDERAL SPONSORED RESEARCH

None.

### PARTIES TO JOINT RESEARCH AGREEMENT

Reactwell, LLC, a Louisiana limited liability company, and employer of Inventors Brandon Iglesias and Yang Song, PhD., at New Orleans, La., and the U.S. Army Combat Capabilities Development Command (CCDC), by Juanita M. Christensen, PhD, and Cindy Wallace, at the Aviation & Missile Center, Redstone Arsenal, Alabama, are parties to a joint research agreement dated Oct. 23, 2019. Applicants are the owners of the Unlimited Ethanol Based Hand Sanitizer as described below, and state, pursuant to 35 U.S.C. 102(c) and 37 CFR 1.104(c)(5)(ii), that: “(A) . . . the subject matter and the claimed invention were made by or on behalf of the parties to a joint research agreement, within the meaning of 35 U.S.C. 100(h) and § 1.9(e), which was in effect on or before the date the claimed invention was made, and that the claimed invention was made as a result of activities undertaken within the scope of the joint research agreement; and (B) The application for patent for the claimed invention discloses or is amended to disclose the names of the parties to the joint research agreement.”

### FIELD OF THE INVENTION

Health care crises, most recently related to the pandemic, bring out the need for hand sanitization to mitigate the spread of contagious viruses and germs. Hand sanitizers are typically ethanol based, liquid disinfectants which are distributed in bottles or on hand wipes, with a finite supply. The bottles or wipes are discarded after use or the bottle contents are exhausted. The novelty of our method of production of an unlimited source of ethanol based, liquid disinfectant, occurs by combining an electrochemical carbon nanospike catalyst with copper, carbon dioxide and water vapor in a chemical process initiated by a power source. We have also improved the common process for making the carbon nanospike—a step in our method of producing an unlimited source of ethanol based, liquid disinfectant—by injection with photons and electromagnetic waves.

The problem we address is medical facilities, and spaces where people congregate, such as malls, stores, sports and concert arenas, are now running out of hand wipes and ethanol based hand sanitizer, which were put in place as a public health convenience to prevent the spread of viruses. We address the problem by teaching a process for producing an unlimited supply of hand sanitizer to be applied directly to human hands or alternatively, to hand wipes, and by teaching the improvement to the common process for making the carbon nanospike.

### BACKGROUND OF THE INVENTION

Oak Ridge National Laboratory (ORNL) reported the discovery of carbon nanospikes (CNS) in 2014, which was an unique morphology of nitrogen-doped graphene comprising 50-80 nm (nanometer) atomically sharp spikes grown using plasma enhanced-chemical vapor deposition (PE-CVD). Due to the atomically sharp texture, 2-3 CNS are able to electrochemically reduce refractory molecules including CO₂ (carbon dioxide) and can generate ethanol when a co-catalyst copper is added.

Low cost, and easily distributable means for converting carbon dioxide into ethanol are well known to those skilled in the art which is referred to as an electrochemical ethanol generator or generation, see Rondinone, US Publication No. 2017-0314148A1, Luo et al, “Facile one-step electrochemical fabrication of a non-enzymatic glucose-selective glassy carbon electrode modified with copper nanoparticles and graphene,” Microchim Acta (2012) Vol. 177, pp. 485-490, and Sheridan et al, “Growth and Electrochemical Characterization of Carbon Nanospike Thin Film Electrodes,” Journal of the Electrochemical Society (2014) Vol. 161, pp. H558-H563.

In 2017, Reactwell, LLC, was awarded an exclusive global license by ORNL to develop an unique scaled up ethanol production process, specifically to improve the technology of the process. Our method of producing a hand sanitizer dispenser that never runs out of product is achieved by combining an improved electrochemical carbon nanospike catalyst with a copper catalyst for ethanol production into a vessel that is fed by a carbon dioxide capture system and a water vapor capture system, triggered by a power source.

The power source is through a potentiostat, which is electronic hardware required to control a three electrode cell. The power source is also well known in the art, with some modifications as tested and described below to accommodate our particular method of production.

### DESCRIPTION OF DRAWINGS

FIG. 1 is a schematic drawing showing state of the art electrochemical ethanol generation from CO₂.

FIG. 2 is a schematic drawing showing detail in the electrochemical ethanol generation from CO₂.

FIG. 3 is a schematic drawing showing novel input of photons and electromagnetic waves to improve the electrochemical ethanol generation from CO₂.

FIG. 4 is a schematic drawing showing detail of a photoelectrochemical ethanol generation from CO₂.

FIG. 5 is a schematic view of a process for supplying power to electrochemical ethanol generation from CO₂.

FIG. 6 is a schematic view of an introduction of photons and electromagnetic waves to a working electrode within a vessel for electrochemical ethanol generation from CO₂.

FIG. 7 is a prospective view of a hand sanitizer dispenser.

### DETAILED DESCRIPTION OF INVENTION

The means for creating ethanol was first described in 2014, see [0007]. The electrochemical ethanol generator produces ethanol from CO₂. The electrochemical ethanol generator is comprised of (1) CO₂ flow, (2) ethanol Chem Chip™, which is a cathode/working electrode, (3) cathode spacer, (4) anion exchange membrane, (5) anode/counter electrode, and (6) anode spacer. In the electrochemical ethanol generator, the ethanol Chem Chip™ is parallel to the counter electrode to achieve a uniform voltage. The spacers contain electrolytes in compartments hosting the ethanol Chem Chip™ and anode/counter electrode.

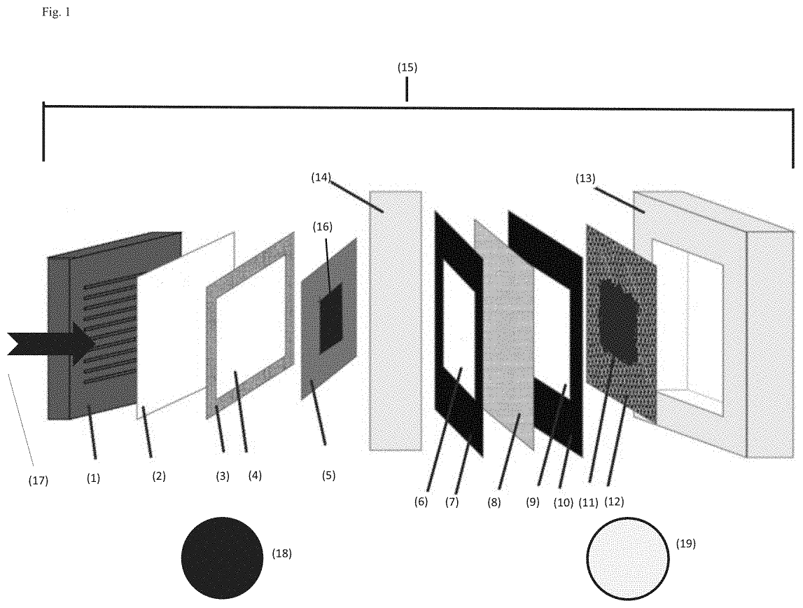

The electrochemical reduction of CO₂ 17 can be carried out in an electrochemical vessel 15, as depicted in FIG. 1. The electrochemical ethanol generator includes an ethanol Chem Chip™ 5 and 16 as a working electrode (cathode), and a counter electrode 11 and 12 (anode), placed in the vessel 15. The counter electrode 11 and 12 may be made of metal such as platinum or nickel. The vessel 15 contains an aqueous solution 18 for the electrolyte and a source of CO₂ 17, meaning an airflow into the vessel 15. The working electrode 5 and 16 and the counter electrode 11 and 12 are electrically connected to each other by virtue of being in contact with the aqueous solution 18. The working electrode 5 and 16 and the counter electrode 11 and 12 only need to be placed in contact with the aqueous solution 18. The vessel also contains an aqueous solution for the anolyte 19. The vessel 15 includes a solid or gel electrolyte membrane 8 (e.g., an anionic exchange membrane) disposed between the working electrode 5 and 16 and the counter electrode 11 and 12. The solid electrolyte membrane 8 divides the vessel 15 into a working electrode compartment housing the working electrode 5 and 16 and a counter electrode compartment housing the counter electrode 11 and 12. The other elements within the vessel 15 are arrayed as depicted in FIG. 1, and include: CO₂ flow-field and current collector 1, micro-porous hydrophobic membranes, such as superhydrophobic PTFE (polytetrafluoroethylene) 2, a first gasket 3, a first flow regime open gasket 4, a second flow regime open gasket 6, a second gasket 7, a third flow regime open gasket 9, a third gasket 10, a first cell spacer for electrolyte 13, and a second cell spacer for electrolyte 14. The process for producing an unlimited source of ethanol based, liquid disinfectant, can be performed on an industrial scale and shipped to customers, or it can be performed locally, in a portable device.

The electrochemical ethanol generator reduces CO₂ to produce ethanol, FIG. 1. The electrochemical ethanol generator is comprised of major components comprising CO₂ flow field 17, micro-porous hydrophobic membranes, working/cathode electrode 5 and 16, anion exchange membrane 8, anode/counter electrode 11 and 12, and cell spacers 13 and 14 for catholyte and anolyte. The major components are separated by gaskets 3, 4, 6, 7, 9, and 10. The flow regime may be open or may contain a membrane. In the electrochemical ethanol generator, the working/cathode electrode 5 and 16 is parallel to the counter/anode electrode 11 and 12 to achieve uniform voltage. The spacers 13 and 14 contain electrolytes in separate compartments which house the working/cathode 5 and 16 and the counter/anode electrode 11 and 12.

The counter electrode 11 and 12 may be composed of a metal such as platinum or nickel, and/or metal oxide such as nickel oxide or ruthenium oxide, and/or mixed metal and/or metal oxide. The electrochemical ethanol generator contains one or two aqueous solution 18 as the electrolytes for the working/cathode 5 and 16 and counter/anode electrodes 11 and 12, and a source of CO₂ 17. The working electrode and the counter electrode are electrically connected to each other by immersion in the aqueous solution 18. The electrochemical ethanol generator includes a solid or gel electrolyte membrane 8 (e.g., anionic exchange membrane) disposed between the working/cathode electrode and the counter/anode electrode. The solid electrolyte membrane divides the electrochemical ethanol generator into a working/cathode electrode compartment housing the working electrode and a counter/anode electrode compartment housing the counter electrode.

The electrochemical ethanol generator or vessel 15 further includes an inlet through which carbon dioxide gas 17 flows into CO₂ flow-field, micro-porous hydrophobic membranes, and reaches the working/cathode electrode 5 and 16. The carbon dioxide gas is made to flow into the vessel 15 at a rate that allows sufficient CO (carbon monoxide) to be transported to the surface of the working electrode. The flow rate of the CO₂ gas is generally dependent on the size of the working electrode. In some embodiments, the flow rate may be about, 3, 10, 30, 50, 70, 90, 100, 120, 140, 160, 180, or 200 mL/min, or within a range bounded by any two of these values. The flow rate varies depending on the size of the working electrode (cathode) and current density. At higher current densities, the same production flow rate of ethanol can be maintained with a smaller relative working electrode surface area. For instance, a flow rate of 3 mL/min is used for a working electrode surface area of 900 cm²; 10 mL/min is used for working electrode surface area of 2700 cm²; and a similar geometric relationship exists for higher flow rates. For larger scale, industrial operations using larger electrodes, the flow rate could be much higher. In some embodiments, before introducing the CO₂ gas into the vessel 15, the CO₂ gas may be humidified with water by passing the gas through a bubbler to minimize the evaporation of the electrolyte. The carbon dioxide being converted may be produced by any known source of carbon dioxide, including the air we breathe.

The source of carbon dioxide may be, for example, a combustion source (e.g., from burning of fossil fuels in an engine or generator), commercial biomass fermenter, or commercial carbon dioxide-methane separation process for gas wells.

The electrochemical ethanol generator efficiently and selectively converts carbon dioxide into ethanol. The ethanol Chem Chip™ 5 and 16 includes carbon nanospikes and copper—containing nanoparticles residing on and/or embedded between the carbon nanospikes. The nanoparticles are well dispersed in the carbon nanospikes. As used herein, the term “nanospikes” refers to tapered, spike-like features present on a surface of a carbon film.

The carbon nanospikes in the Chem Chip™ electrocatalyst can have variable lengths. The carbon nanospikes on the electrodes can be layered to increase current density. Generally, the nanospike length may be precisely or about, 30, 35, 40, 45, 50, 55, 60, 65, 70, 75, 80, 85, or 90 nm, or within a range bounded by any two of these values. In particular embodiments, the carbon nanospikes have a length from about 50 to 80 nm.

At least a portion (e.g. at least 30, 40, 50, 60, 70, 80, or 90%) of the carbon nanospikes in the electrocatalyst is composed of layers of puckered carbon ending in a straight or curled tip. The width and taper of the nanospike tips as well as the curling dimensions determine the electric field and angle for the reaction. Sharper tips result in an increased ethanol yield. The width of the straight or curled tip may be precisely or about, 0.5, 0.6, 0.7, 0.8, 1.0, 1.1, 1.2, 1.3, 1.4, 1.5, 1.6, 1.7, 1.8, 1.9, 2.0, 2.1, 2.2, 2.3, 2.4, or 2.5 nm, or within a range bounded by any two of these values. In particular embodiments, the straight or curled tip has a width of from about 1.8 to 2.2 nm. Ethanol yield can also be increased by hydrophobicity (property of repelling water) of the straight or curled tips.

The carbon nanospikes are doped with a dopant selected from nitrogen, boron, or phosphorous. The dopant prevents well-ordered stacking of carbon, thus promoting the formation of a disordered nanospike structure. Without doping, the production of ethanol is not observed. In one embodiment, the carbon nanospikes are doped with N (nitrogen). Doping with N varies. The amount of the dopant in the carbon nanospikes may be precisely or about, 3, 4, 5, 6, 7, 8, or 9 atomic percentages, or within a range bounded by any two of these values. In particular embodiments, the dopant concentration is from about 4 to 6 atomic percentages.

The carbon nanospikes can be prepared by any method known to those skilled in the art. In a first embodiment, the carbon nanospikes can be formed on a substrate by PE-CVD with any suitable carbon source and dopant source. In a second embodiment, the substrate is a semiconductive substrate. Some examples of semiconductive substrates include silicon, germanium, silicon germanium, silicon carbide, and silicon germanium carbide. In a third embodiment, the substrate is a metal substrate. Some examples of metal substrates include copper, cobalt, nickel, zinc, palladium, platinum, gold, ruthenium, molybdenum, tantalum, rhodium, stainless steel, and alloys thereof. In a fourth embodiment, an arsenic-doped (As-doped) silicon substrate is employed and nitrogen-doped carbon nanospikes are grown on the As-doped silicon substrate using acetylene as the carbon source and ammonia as the dopant source.

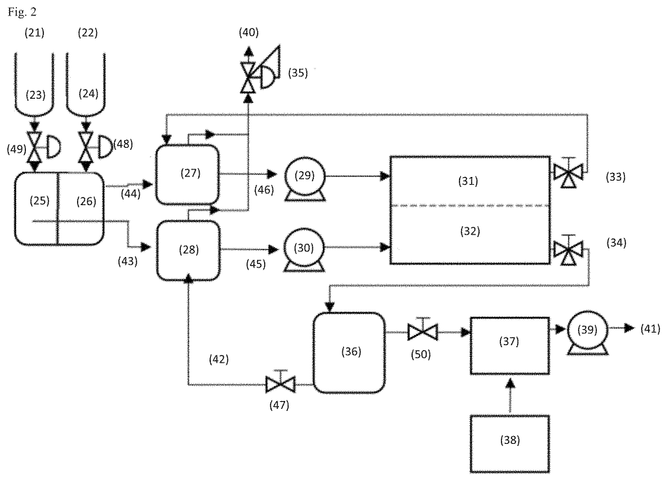

A more detailed explanation of the electrochemical ethanol generation from CO₂ is shown in FIG. 2. Air from an external source containing carbon dioxide enters the vessel 21. Simultaneously, water vapor in the air from an external source enters the vessel 22. A CO₂ concentrator system 23 provides means for adsorbing and crystallizing the CO₂. An H₂O (water) deionizer system 24 provides means for obtaining pure water. A flow controller and meter 25 monitors CO₂ input. A flow controller and meter 26 monitors H₂O input. A first reservoir 27 stores the anode electrolyte and H₂O. A second reservoir 28 stores the cathode electrolyte and CO₂. An injection pump 29 pushes the anolyte (anode's electrolyte+H₂O) into the vessel. An injection pump 30 or compressor pushes the catholyte (cathode's electrolyte+CO₂) into the vessel. The electrolysis flow cell anolyte compartment 31 has temperature and pressure sensors, analyzers, and membranes separating it from catholyte chamber 32. The electrolysis flow cell catholyte compartment 32 has temperature and pressure sensors, analyzers, and membranes separating it from anolyte chamber in 31. A control valve 33 for acidic/basic control and concentration control monitors and preserves the integrity of the water input. A control valve 34 for acidic/basic control and concentration control monitors and preserves the integrity of the carbon dioxide. A backpressure regulator 35 prevents back flow into the vessel. A gas/liquid membrane separator 36 separates the elements. A blending reservoir 37 contains and dispenses ethanol, water and additives at target percentage of volume, and also contains analyzers and sensors for ethanol volume, temperature and pressure measurements. Addition of H₂O₂ (hydrogen peroxide) 38 from a separate electrochemical unit or storage reservoir as well as blending with H₂O achieves a target volume of 70% ethanol in the hand sanitizer product. Aromatic scents and antimicrobial additives as well as antimicrobial and antiviral particles may also be added here. A dispensing pump with spray nozzle 39 yields the hand sanitizer 41 at volume of 70% ethanol in water, and can be blended for finished product dispensing directly onto a person's hand or onto hand wipes. A vent 40 can have a carbon filter added to remove any byproducts prior to discharge into external air. In an embodiment an electrolyte recirculation loop 42 may be used to improve quality of the finished product. In another embodiment the carbon dioxide flow line 43 into the catholyte circulation loop, can have filters and analyzers/sensors on it as well, for improved quality of finished product. In another embodiment the water flow line 44 into the anolyte circulation loop, can have filters and analyzers/sensors for the same reason. A catholyte with recirculation flow 45 may be included on the CO₂ line. An anolyte with recirculation flow 46 may be included on the H₂O line. A backpressure regulator 47 for the cathode sub-system, prevents back flow. A manual valve 48 for isolation and replacement of a water module may be included. And, a manual valve 49 for isolation and replacement of a carbon dioxide module may be included.

Explaining the operations in FIG. 2, CO₂ being converted is captured from surrounding air and concentrated. Water is captured from surrounding air or from a tap source and added after purification by H₂O deionizer, as compensation for an aqueous electrolyte solution to maintain the desired concentration level. The flow rate of H₂O to an anolyte reservoir and to a catholyte reservoir is regulated by H₂O flow controller. The flow rate of CO₂ to catholyte reservoir is regulated by CO₂ flow controller. The anolyte in the anode compartment is injected and circulated by an injection pump and a control valve for anolyte. The catholyte in the cathode compartment is injected and circulated by injection pump and a control valve for catholyte. The acidic/basic of anolyte and catholyte are monitored by built-in sensors. The pressure of electrolyte is monitored by backpressure regulator. The electrochemical generated ethanol in catholyte is separated by gas/liquid separation membrane, and stored in a blending reservoir for dispensing, where the blending reservoir contains ethanol and water. The catholyte after separation is returned to the catholyte reservoir via the catholyte recirculation loop with backpressure regulator. Carbon dioxide flow and water flow into the circulation loop also have additional filters and analyzers/sensors. H₂O₂ is added from a separate electrochemical unit or storage reservoir as well as blended with H₂O for target volume of 70% ethanol in hand sanitizer product. The hand sanitizer product can also have scents and antimicrobial additives as well as antimicrobial and/or antiviral particles added. The hand sanitizer product from the ethanol generator with optional additives is dispensed by a dispensing pump with a spray nozzle.

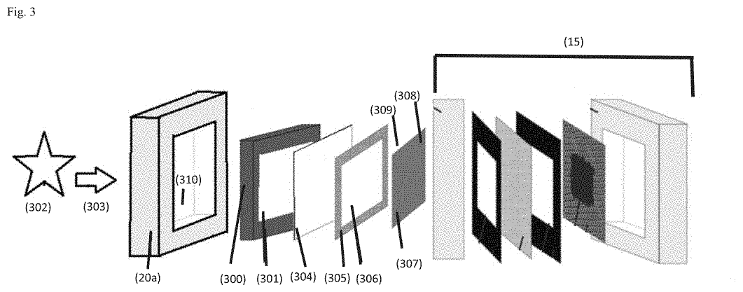

An improved method for making the carbon nanospike, is through injection with photons and electromagnetic waves, as depicted in FIG. 3. A photochemical ethanol generator as shown here, is an improvement on the electrochemical ethanol generator described in FIG. 1. In FIG. 3 method, a CO₂ flow field and current collector 300 with an open center 301 allows the passage of white light (photons) and electromagnetic spectrum 302 at higher and lower frequency than white light in a vessel. Micro-porous hydrophobic membranes 304, such as superhydrophobic PTFE, allow photons to pass through. A gasket 305 permits photons to pass around. A flow regime gasket 306 (open or membrane), allows photons to pass through. A carbon nanospikes 307 on carbon paper or nitrogen or phosphorous silicon wafer substrate with copper or other alloys and a Ni/Cu/Ti/Ru/Co/Pd gas diffusion layer permit generation of ethanol. Here photons and electrons interact to enable higher current densities than previously considered possible, with plasmonic and thermal effects. Addition of nanoparticles and tubes attached to the back side enhance conductivity. A photon injection manifold 308 obtains blocked nanoparticles and carbon nanospikes within its layered structure. Metals, alloys and hydrophobic coatings 309 are placed on a topside of the carbon nanospike on substrate carbon paper. A source of photons and electromagnetic waves, coherent, laser or other light source 302 enters the photochemical ethanol generator. Photons and electromagnetic waves are introduced along with carbon dioxide flow 303. Quartz, glass or other transparent material 310 is used to direct the photons into an enclosed cell spacer, where a gas is contained but light passes through nearly unaffected onto the surface of the carbon nanospike. Light is enabled to pass into a cell spacer 20a through the CO₂ in gas or in liquid phase.

Explaining the role and interaction of the components in FIG. 3, the photoelectrochemical ethanol generator is improved from the electrochemical ethanol generator FIG. 1, by modifying the CO₂ flow-field to a CO₂ flow-field and a current collector with an open center 300, enabling light to pass through a micro-porous membrane 301, and light enabling passage cell spacer 20a enclosed with quartz, glass or other material 310 that is transparent to photons so as to allow the passage of white light (photons) and electromagnetic spectrum 302 at higher and lower frequency than white light. Other major components in the photoelectrochemical ethanol generator FIG. 3 are similar to that in the electrochemical ethanol generator FIG. 1, including the working/cathode electrode 5 and 16, anion exchange membrane 8, counter/anode electrode 11 and 12, and cell spacers for catholyte and anolyte 13 and 14, within a vessel 15. The major components are separated by gaskets 3, 4, 6, 7, 9, and 10. The flow regime may be open or with a membrane. As in the electrochemical ethanol generator, the working/cathode electrode 5 and 16 is parallel to the counter/anode electrode 11 and 12 to achieve a uniform voltage. The spacers 13 and 14 contain electrolytes in compartments hosting the working/cathode 5 and 16, and the anode/counter electrode 11 and 12.

Continuing with the explanation from FIG. 3, as in FIG. 1, the counter electrode 11 and 12 may be same as or it may be different than the counter electrode used in electrochemical ethanol generator, and be comprised of a metal such as platinum or nickel, and/or metal oxide such as nickel oxide or ruthenium oxide, and/or mixed metal and/or metal oxide. Similar to the electrochemical ethanol generator FIG. 1, the photoelectrochemical ethanol generator FIG. 3 contains one or two aqueous solution as the electrolytes for the working/cathode and counter/anode electrodes and a source of CO₂. The working electrode and the counter electrode are electrically connected to each other and in contact with the aqueous solution. The working electrode and the counter electrode only need to be placed in contact with the aqueous solution. The electrochemical ethanol generator includes a solid or gel electrolyte membrane (e.g., anionic exchange membrane) disposed between the working/cathode electrode and the counter/anode electrode. The solid electrolyte membrane divides the electrochemical ethanol generator into a working/cathode electrode compartment housing the working electrode and a counter electrode compartment housing the counter electrode. The process for producing an unlimited source of ethanol based, liquid disinfectant, by the photoelectrochemical ethanol generator can also be performed on an industrial scale and shipped to customers, or it can be performed locally, in a portable device.

And, continuing with the explanation from FIG. 3, the photoelectrochemical ethanol generator includes an inlet through which carbon dioxide gas flows into the CO₂ flow-field 303, 300, and 310, micro-porous hydrophobic membranes, and reaches the working/cathode electrode. The carbon dioxide gas is made to flow into the photoelectrochemical ethanol generator at a rate that allows sufficient CO, to be transported to the surface of the working electrode. The flow rate of the CO₂ gas is generally dependent on the size of the working electrode and may be tuned due to the applied photons and electromagnetic spectrum. In some embodiments, the flow rate may be about 3, 10, 30, 50, 70, 90, 100, 120, 140, 160, 180, or 200 mL/min, or within a range bounded by any two of these values. However, for larger scale, industrial operations using larger electrodes, the flow rate could be much higher, involving tons per minute. In some embodiments, before introducing the CO₂ gas into the photoelectrochemical ethanol generator, the CO₂ gas may be humidified with water by passing the gas through a bubbler to minimize the evaporation of the electrolyte. The carbon dioxide being converted may be produced from any known source of carbon dioxide.

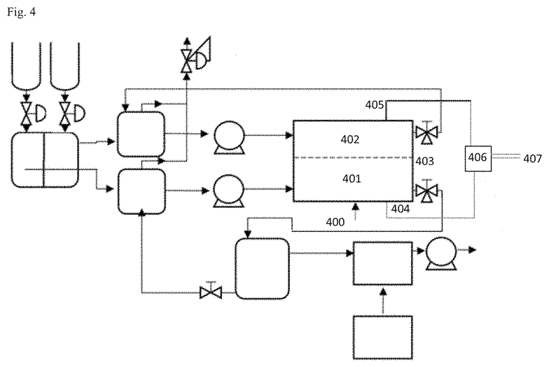

In FIG. 4, the detail for the photoelectrochemical ethanol generator is provided. An input light source and injection for CO₂ flow-field and current collector with an open center 400 to allow the passage of white light (photons) and electromagnetic spectrum at higher and lower frequency than white light initiates the process. A cathode 401 sits in a first chamber, where the first chamber contains micro-porous hydrophobic membranes, such as superhydrophobic PTFE, and where photons pass through a first gasket, photons pass around a second gasket, continuing through a flow regime gasket (open or membrane), then pass through the carbon nanospikes on carbon paper or nitrogen or phosphorous silicon wafer substrate with copper or alloys and a Ni/Cu/Ti/Ru/Co/Pd gas diffusion layer for generation of ethanol. In a first chamber with the cathode 401, photons and electrons interact to enable higher current densities than previously considered possible, along with plasmonic and thermal effects. An anode 402 sits in a second chamber, where the second chamber receives the photons and electrons through a first flow regime gasket (open or membrane), a first gasket, an alkaline membrane (AEM), a second flow regime gasket (open or membrane), a second gasket, a counter electrode (anode) for generation of O2 or H₂O₂ (treatment 2), a counter electrode (anode) for generation of O2 or H₂O₂ (surface treatment 1), and a first cell spacer for the electrolyte. A membrane 403 separates the first and second chambers. The power input to the cathode is comprised of a DC power source 404. The power input to the anode is comprised of a DC power source 405. A rectifier (to convert AC from a wall outlet to DC) 406 may be used where only AC is available. Where input power is DC 407 then the rectifier is not required and can be bypassed or simply not built in.

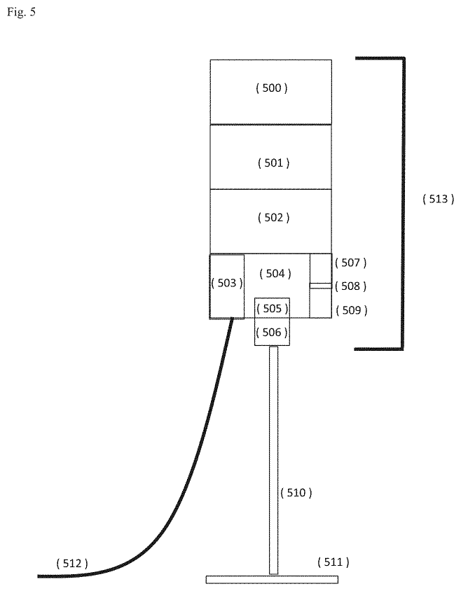

In FIG. 5 the roles of additional components are explained: An atmospheric water capture subsystem 500, pulls water from air within a building or environment where the hand sanitizer product is located. Cartridges with sorbent material are modular, and cartridge based like an inkjet printer to permit easy replacement. A carbon dioxide capture subsystem 501, pulls carbon dioxide from air in the building or environment, in series or in parallel with atmospheric water capture subsystem 500. An electrochemical converter 502 with potentiostat controls is fed by DC power 512. A reservoir 504 for sanitizer with volume of 70% ethanol and water contains and disperses product. A sensor and a button 505 is provided for dispensing touchless product. Should the sensor fail, then a fail over button permits dispensing the product. In an embodiment, the sensor can also perform facial recognition and biometric analysis for virus, bacteria and pathogens. A dispenser 506 sprays sanitizer in mist or stream or gel form onto a target, such as a human hand or hand wipe. In another embodiment, during the manufacture of hand wipes, the hand wipes can be dipped in the 70% ethanol solution which is made by either the electrochemical ethanol generator or the photoelectrochemical ethanol generator. A rectifier 503 receives AC power and converts it to DC power and routs power to an electronic system for carbon dioxide capture. The process provides atmospheric water capture, electrochemical conversion for ethanol, electrochemical conversion for hydrogen peroxide, as well as photoelectrochemical conversion for ethanol and permits mixing/flowing of fluids within an endless sanitizer dispenser 508. If power input is DC then this module serves to condition the DC power voltage and amperage regulation prior to feeding it into the subsystem components. In an embodiment, a replaceable cartridge for scents or gel agent 507 is available.

Continuing on FIG. 5, in an embodiment, a replaceable cartridge for bittermint 509 (sour taste) is added to sanitizer so people will not be tempted to ingest the hand sanitizer product. In another embodiment, a cartridge for IoT connectivity transducer, wifi, Bluetooth, and/or cellular access is available, to communicate the number of sanitizer users per day, and biometric data to permit further analysis on effectiveness of the product. On premises or off premises security configuration can permit linkage to electric prices to assess costs and inventory for replacement cartridges. A stand 510 can house additional carbon dioxide and atmospheric water capture sorbent or additional electrochemical stacks with optional cells. A baseplate 511 for the stand is optional, can store captured elemental carbon for recycling or soil enrichment. And, a power cord for AC or DC is available.

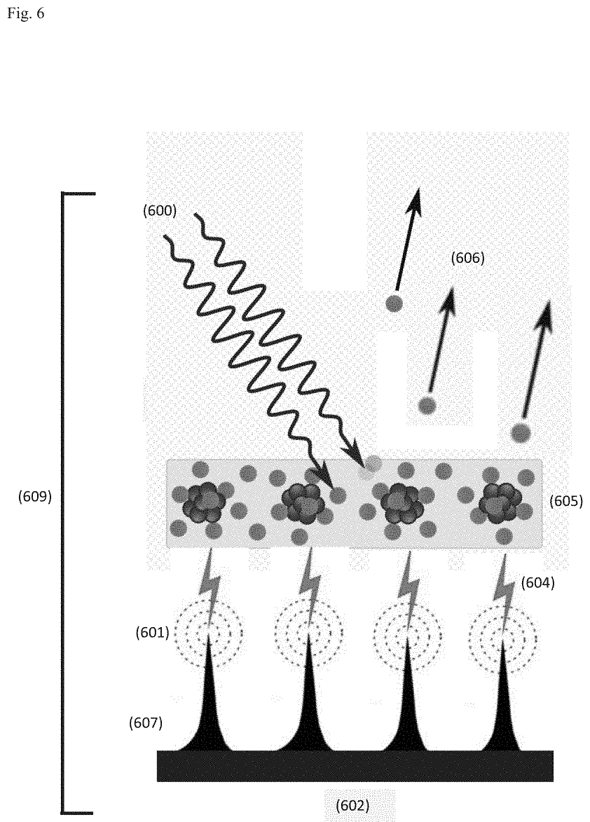

Explaining the reaction which occurs when photons and electromagnetic waves are used to enhance the result, we turn to FIG. 6. Photons and electromagnetic waves 600 enter the surface of the cathode from a light source or electromagnetic wave (EM) source. Electrons 601 enter the carbon nanospike from substrate source 602. Photons 608 enter the carbon nanospike wave function at the tip of carbon nanospike (nanoscale tip) where metals and alloys on the carbon nanospike tip are doped with nitrogen and hydrophobic coatings. Electrons 604 enter the tip of the carbon nanospike and exhibit field effects, such that the work function is nearly undetectable. The photons and electrons interact coherently 605, such that higher energy reactions are required to quantize the energy levels. This is accomplished by directly injecting electrons with raw carbon nanospike tips for N2+H2O to NH3 reactions and as for CO2+H2O to C2+ reactions. Due to the higher energy state, the injection of photons or EM (electromagnetic) wave to the electrochemical cathode surface suppresses hydrogen evolution reaction, which enables targeted and selective production of ethanol and other more desirable compounds. For example in a CO2 and H2O environment the reaction favors C2+ molecules, but in a N2 and H2O environment the reaction favors ammonia production (NH3).

Continuing on FIG. 6, electrons 606 from carbon nanospike tips and interactions with metal/alloy/actinide/photon at the tip of carbon nanospikes are available for reaction and injection to form complex molecules such as C2+ or NH3 triple bond molecules. The photoelectrochemical cathode 607 surface is used for suppression of hydrogen evolution reaction and selective formation of C2 and NH3 triple bond molecules based upon the environment, comprising CO2, N2, and H2O in a catholyte. The catholyte enters at the substrate source 602 and the CO2 or N2 is on the cathode chamber 608. The cathode chamber 608 is exposed to CO2 or N2 and electromagnetic waves/photons. The substrate source 602 is comprised of carbon paper, a nitrogen/phosphorous silicon semi-conductor that serves as a conductor for electrons into carbon nanospikes, and where multiple treatments on the surface and base are intended to maximize electron flow and prevent catholyte leakage into the environment 608. Electrolyte transits into membrane between 602 and 608 is comprised of CNS with PTFE, which enables efficient reaction to maximize current density and stability over time. Where time is defined as one day. A photoelectrochemical cell 609 comprised of carbon nanospike on substrate with various surface treatments such as doping, impregnation with metals/alloys/actinides and such useful improvements to the carbon nanospike to maximize current density and enable photoelectrochemical reactions to proceed, is selective for C2+ molecules and N triple bond based molecules. Water is added directly to the system. Water enters the system from 602 in catholyte as well as in moisture addition 608 for the CO2 and N2 environment.

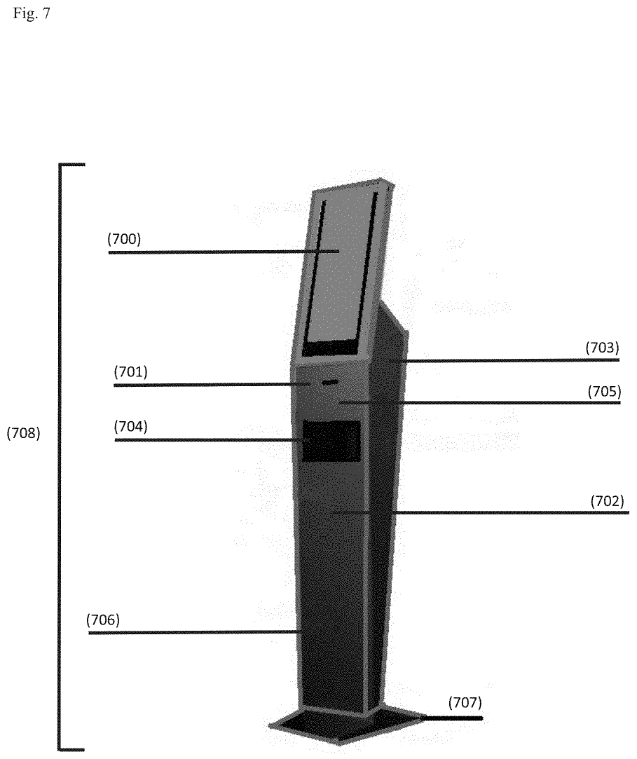

The hand sanitizer product is dispensed from a device, FIG. 7. In one embodiment, the device is free-standing and can be accessed by one person at a time. In another embodiment, a single device may permit access by more than one person. A display screen 700 on the device can be used for educational messages or for marketing messages by an organization. The display screen receives input from sensors 702 and 701. In an embodiment, the device can be tailored to provide precise content delivery through cloud or off premise based sensory or biometric data, such as a person's temperature, blood pressure, facial recognition, and/or emotional recognition, etc. A carbon dioxide capture subsystem 703, an atmospheric water capture subsystem, H2O reservoir, CO2 reservoir, and ethanol reservoir are located on the device. Sensor and interface 701 for removal of replacement cartridges (for CO2 sorbent, H2O sorbent, scent, bittermint, and other optional characteristics), work with displays 700, and also serve as a two point check system prior for dispensing hand sanitizer 704. Cartridge 705 access points in front and back, replacement for CO2 sorbent, H2O sorbent and consumables (bittermint, scent, gel additive), are located on the device. A button in case touchless sensor fails is also provided. A “Touchless” sanitizer 704 dispenser with built in sensor is on the device. Access points, sensors and a pump station 702 for linking the electrochemical stacks with H2O, CO2 inlet and ethanol outlet, also house the electrical, controls and photonics systems. Electrochemical stacks 706 and photoelectrochemical stacks for conversion of CO2 and H2O into ethanol as well as electrolyte management systems for catholyte and anolyte are at the base of the device. A power cord 707 and a rectifier for changing AC input to DC output is provided, although, the device can also work with DC power directly.

Concluding the discussion of FIG. 7, an endless sanitizer integrated system 708 is comprised of carbon dioxide capture, atmospheric water capture, electrochemical/photoelectrochemical stacks, potentiostat, touchless dispenser, and a replacement management systems for consumable items in the process, interfaces with cloud or on premises for sensors, facial recognition, precision content delivery and analytics for use of the system in facility administration (number of persons using the sanitizer per day, number of hands sanitized per day, average temperature of person submitting to sanitization, facial recognition, viral load detection, bacterial detection, and various other uses of said hardware and software). Input voltage is 110/120 VAC or 208/277 VAC or DC. Current load is sized for average plug 15 amp to 20 amp, frequency at 60 Hz or 50 Hz, plug and cord for wall outlet NEMA1-5 or other specifications. The device can work in environmental conditions indoors or be ruggedized for outdoor use. Temperature indoor range is 40 to 90 deg F. Temperature outdoor range is −30 deg F. to 200 deg F. Relative humidity indoor >25%. Production capacity is 1 liter per day of hand sanitizer at volume 70% ethanol, with H₂O₂ potential for generation on anode and ethanol on cathode for WHO specifications.

We claim:

1. A process for providing unlimited source of ethanol based, liquid disinfectant for use directly onto human hands or onto hand wipes, comprising:
placing a carbon nanospike catalyst which is doped with nitrogen and a copper catalyst in a vessel;
providing a source into the vessel for carbon dioxide, water and water vapor;
providing a means for an electrochemical process to create ethanol which is initiated by a means for a power source in the vessel; and,
wherein the ethanol can be turned into a supply of liquid hand sanitizer.

2. The process of claim 1, wherein the ethanol can be turned into a liquid to coat hand wipes.

3. The process of claim 1, wherein the process can occur in a portable vessel.

4. A process for providing unlimited source of ethanol based, liquid disinfectant for use directly onto human hands or onto hand wipes, comprising:
placing a carbon nanospike catalyst which is doped with nitrogen and a copper catalyst in a vessel;
providing a source into the vessel for carbon dioxide, water and water vapor;
injecting photons and electromagnetic waves into the vessel;
providing a means for an electrochemical process to create ethanol which is initiated by a means for a power source in the vessel; and,
wherein the ethanol can be turned into a supply of liquid hand sanitizer.

5. The process of claim 4, wherein the ethanol can be turned into a liquid to coat hand wipes.

6. The process of claim 4, wherein the process can occur in a portable vessel.

7. A process for providing unlimited duration energy storage of ammonia gas, liquid or mixed phase of gas and liquid, comprising:
placing a carbon nanospike catalyst which is doped with nitrogen, metals and actinides in a vessel;
providing a source for nitrogen, water and water vapor into the vessel;
providing a means for an electrochemical process to create ammonia which is initiated by a means for a power source in the vessel; and,
wherein the ammonia can be turned into an energy storage for the storage of electrons in ammonia (NH3) bonds as well as a reverse reaction to release electrons, fertilizer or fuel.

8. The process of claim 7, wherein the ammonia can be turned into urea by addition of carbon dioxide.

9. The process of claim 7, wherein the process can occur in a portable vessel.

10. A process for providing unlimited duration energy storage, of ammonia gas, liquid or mixed phase of gas and liquid, comprising:
placing a carbon nanospike catalyst which is doped with nitrogen, titanium, uranium and alloys catalyst in a vessel;
providing a source into the vessel for nitrogen, water and water vapor;
injecting photons and electromagnetic waves into the vessel;
providing a means for a single-step photoelectrochemical process to create ammonia which is initiated by means of a power source in the vessel; and,
wherein the ammonia can be turned into a supply of energy storage for the storage of electrons in NH3 bonds as well as a reverse reaction to release electrons, fertilizer or fuel.

11. The process of claim 10, wherein the ammonia can be turned into urea by addition of carbon dioxide.

12. The process of claim 10, wherein the process can occur in a portable vessel or hard wired to building supply or automotive, truck or generator, for in-situ diesel exhaust fluid generation.

13. A process for providing unlimited hydrogen peroxide, comprising:
placing a carbon nanospike catalyst which is doped with nitrogen, Tungsten metals and actinides in a vessel;
providing a source into the vessel for carbon dioxide, water and water vapor;
injecting photons and electromagnetic waves into the vessel;
providing a means for an electrochemical process to create hydrogen peroxide (H2O2), on anode/anolyte, which is initiated by a means for a power source in the vessel; and,
wherein the hydrogen peroxide can be utilized in hand sanitizer.

14. A process for providing unlimited hydrogen peroxide, comprising:
placing a carbon nanospike catalyst which is doped with nitrogen, Tungsten metals and actinides in a vessel;
providing a source into the vessel for nitrogen, water and water vapor;
injecting photons and electromagnetic waves into the vessel;
providing a means for an electrochemical process to create hydrogen peroxide (H2O2), on anode/anolyte, which is initiated by a means for a power source in the vessel; and,
wherein the hydrogen peroxide can be utilized in hand sanitizer.
